(19) United States
(12) Patent Application Publication
(10) Pub. No.: US 2011/0088456 A1
(43) Pub. Date: Apr. 21, 2011

(54) NORMALIZED HYDROGEN SENSING AND METHODS OF FABRICATING A NORMALIZED HYDROGEN SENSOR

(76) Inventors: FAN REN, Gainesville, FL (US); Stephen John Pearton, Gainesville, FL (US)

(21) Appl. No.: 12/723,802

(22) Filed: Mar. 15, 2010

Related U.S. Application Data
(60) Provisional application No. 61/252,437, filed on Oct. 16, 2009.

(51) Int. Cl. G01N 27/00; H01L 29/778

(52) U.S. Cl. 73/31.06; 257/194; 257/253; 257/E29.246

(57) ABSTRACT

HEMT-based hydrogen sensors are provided. In accordance with one embodiment, a normalized sensor is provided having a control HEMT-based sensor connected in series to an active HEMT-based sensor. The control and the active sensor include functionalized gate regions. The gate functionalization for both the control and the active sensor is the same material that selectively absorbs hydrogen gas. The control sensor further includes a protective layer to inhibit its gate functionalization from being exposed to hydrogen. In one embodiment, the final metal for the contacts of the sensors is used as the protective layer. In other embodiments, the protective layer is a dielectric or polymer.

### CROSS-REFERENCE TO RELATED APPLICATION

This application claims the benefit of U.S. Provisional Application Ser. No. 61/252,437, filed Oct. 16, 2009, which is hereby incorporated by reference in its entirety, including all figures, tables and drawings.

### BACKGROUND OF THE INVENTION

Presently, interest and research has been focusing on the ability to adequately detect the presence of hydrogen. In particular, with the emergence of hydrogen-fueled applications, including hydrogen-fueled automobiles and proton-exchange membrane (PEM) and solid oxide fuel cells for space craft, and other long-term sensing applications, the ability to detect or sense hydrogen in these environments has become a challenge. Specifically, these sensors are generally required to selectively detect hydrogen from H₂S, CH₄, CO, humidity, and volatile organic compounds at the temperature ranging from −40° C. to 80° C. with minimal power consumption and weight and with a low rate of false alarms. The hydrogen sensors may be used as safety devices to detect hydrogen gas leakage or as hydrogen monitoring devices to monitor normal storage/converter operation. In addition to fuel cell applications, hydrogen sensors may be used in applications where hydrogen is an undesirable contaminant.

There are a variety of hydrogen sensors using different mechanisms to detect the gas. A popular approach involves using palladium (Pd) to selectively absorb hydrogen gas and form palladium hydride resulting in a change of the Pd conductivity. Unfortunately, many palladium-based sensors tend to have a strong temperature dependence, which can cause slow response times at very low temperatures. These sensors are typically based on direct conductivity measurement. There is no amplification effect of the detecting signal. Once the hydrogen sensing response slows down, the sensor is not sensitive enough to effectively detect the hydrogen. Therefore research has been conducted to provide sensors capable of proper function at room temperature.

### BRIEF SUMMARY

Embodiments of the present invention relate to methods and devices for hydrogen sensing. A normalized HEMT-based hydrogen sensor is provided.

In one embodiment an HEMT is functionalized with a material that can selectively absorb hydrogen gas. In an exemplary embodiment, the HEMT is an AlGaN/GaN HEMT. According to certain embodiments, the material being used to selectively absorb hydrogen gas is palladium, platinum, palladium/oxide, platinum/oxide, LaNi₅, or LaNi₅/oxide. For embodiments incorporating oxide as part of the material being used to selectively absorb hydrogen gas, the oxide can be, for example, Sc₂O₃, HfO₂, Al₂O₃, Gd₂O₃, GGO (gallium gadolinium oxide), GGG (gallium gadolinium garnet), TiO₂, SiO₂, SiO, or SiNx.

In further embodiments, a normalized design utilizing a control and an active sensor having a same functionalization is provided to improve device readings.

### BRIEF DESCRIPTION OF THE DRAWINGS

FIG. 1A shows a schematic diagram of a differential amplifier circuit that can be used to output the signal from the response of a normalized temperature sensor in accordance with an embodiment of the present invention.

FIG. 1B shows a plan view schematic representation of contact pads for a normalized sensing device in accordance with an embodiment of the present invention.

FIG. 2 shows a cross-sectional view of a normalized hydrogen sensor according to an embodiment of the present invention.

FIGS. 3A-3F show process views for explaining a method of fabricating a hydrogen sensor according to an embodiment of the present invention.

FIG. 4 shows a cross-sectional view of a normalized MOS-HEMT based hydrogen sensor according to one embodiment of the present invention.

FIGS. 5A-5G show process views for explaining a method of fabricating a hydrogen sensor according to another embodiment of the present invention.

FIG. 6 shows a plot of diode current vs. bias voltage comparing a related art differential sensor showing the sensitivity of the device to temperature and bias to a normalized hydrogen sensor in accordance with an embodiment of the present invention.

FIG. 7 shows a cross-sectional view of a LaNi₅ gated nitride based HEMT hydrogen sensor according to one embodiment of the present invention.

### DETAILED DISCLOSURE

Embodiments of the present invention relate to high electron mobility transistor (HEMT) based sensors. According to an embodiment, differentially arranged HEMTs can be utilized to improve consistent device readings. In a specific embodiment, a hydrogen sensor is provided.

According to an embodiment of the present invention, an AlGaN/GaN HEMT can be used for hydrogen detection. However, embodiments are not limited thereto. For example, other HEMTs such as AlGaN/InGaN/GaN, AlN/GaN, AlN/InGaN/GaN, AlGaAs/GaAs, AlGaAs/InGaAs, InAlAs/InGaAs, and InGaP/GaAs single or double heterojunction HEMTs can be used for hydrogen detection. The sensing component, or active device, can be a gate-functionalized HEMT. In certain embodiments, Pd may be used for hydrogen sensing by serving as the gate functionalization material. In one embodiment, instead of Pd, platinum (Pt) is coated on the gate region of the HEMT to enhance catalytic dissociation of molecular hydrogen. In a further embodiment, an oxide is further included on the gate region below the Pt or Pd. The oxide may be a metal oxide. In another embodiment, LaNi₅ can be used for hydrogen detection, where the LaNi₅ is integrated with a nitride HEMT for hydrogen sensing applications. In a further embodiment, LaNi₅/oxide may serve as the gate functionalization material.

According to an embodiment of the present invention, the HEMT can be configured as a Schottky diode-type gas sensor where the gate region employs a material that can selectively absorb hydrogen, which lowers the Schottky energy barrier. However, advantageously, compared to Schottky diodes, HEMTs can have much higher sensitivity because they are true transistors and therefore operate with gain. In addition, a MOS-gate version of the HEMT can provide an improved thermal stability as compared to a metal-gate structure. When exposed to changes in ambient environment, changes in the surface potential leads to large changes in channel current.

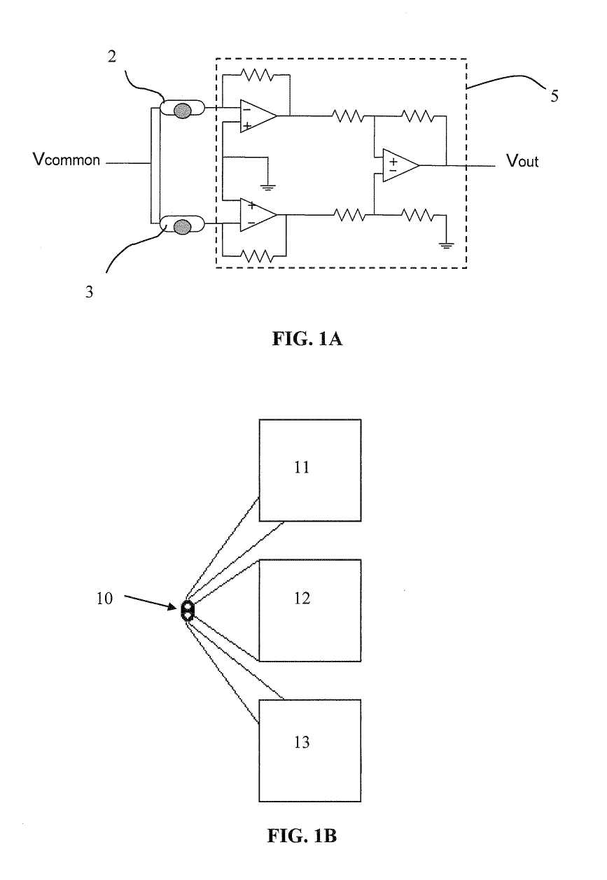

An improved temperature response sensor is provided that utilizes a normalized diode or field effect transistor (FET) configuration. According to an embodiment, a control sensor and an active sensor are arranged in a common ground configuration. A differential amplifier can be connected to the normalized hydrogen sensor (e.g., the combination of the control sensor and the active sensor) to provide an amplified output of the sensor's response to hydrogen in the ambient environment. For example, FIG. 1A shows a schematic representation of a normalized hydrogen sensor output circuit 5 in accordance with one embodiment of the present invention. In other embodiments, the schematic shown in FIG. 1A can be replaced with any suitable differential amplifier circuit. As shown in FIG. 1A, the control sensor 2 is connected to one input of the output circuit 5 and the active sensor 3 is connected to another input of the output circuit 5. The control sensor 2 and the active sensor 3 are also connected to a common node providing a common voltage V_{common}. Though not shown in the figures, an initialization circuit can be included to reset the sensor and/or to bias the output circuit 5. Any suitable biasing circuit can be used. FIG. 1B shows contact pads for the sensor 10. A first contact pad 11 can connect the control sensor to the differential amplifier circuit; a second contact pad 12 can connect the common node of the control sensor and the active sensor to a ground signal; and a third contact pad 13 can connect the active sensor to the differential amplifier circuit.

In accordance with embodiments of the present invention, both the control and the active sensor are exposed to the ambient temperature. In contrast with certain related art sensors using a reference device, the control sensor of an embodiment of the present invention has the exact same gate metal to semiconductor interface as the active sensor of the subject device. However, for the subject device, the gate metal of the control sensor is covered with another metal, dielectric, or polymer, which inhibits the gate metal of the control sensor from being exposed to the gas in the surrounding environment and/or is inert to the hydrogen in the surrounding environment. The sensing response signal is output from the potential difference between the control sensor and the active sensor. Common mode rejection can be used to reject the signal that is common to the inputs (i.e. remove temperature effects found common in both the control sensor and the active sensor signals). The source regions of the sensors are grounded together for the diode mode sensing and the drain regions of the sensor are floated. If the FET mode is used for the sensing, the drain current or threshold voltage of the HEMT will be used to monitor the hydrogen concentration instead of diode current used in the diode mode sensing.

The normalized configuration provides a built-in control diode to reduce false alarms due to temperature swings or voltage transients. Since both the control and the active sensor have the same gate metal (or gate oxide) to semiconductor interface, the diode or FET characteristics will be the same regardless of ambient temperature. Thus, the differences in diode or FET characteristics for the two sensors (control and active) occur only in their exposure to the hydrogen ambient. Specifically, the active sensor will respond to the hydrogen and the control sensor will not. The HEMT will amplify the signal detected from the gate metal of the active sensor, thereby enabling extremely sensitive sensing. The amplified signal of the active sensor can then be compared to the control sensor signal through, for example, the differential amplifier circuit of FIG. 1A to provide a normalized signal. In addition, embodiments of the present invention can accomplish these reductions in false alarms at a wide range of temperatures. In certain embodiments, the subject sensor can reduce false alarms for temperatures between −40° C. and 80° C.

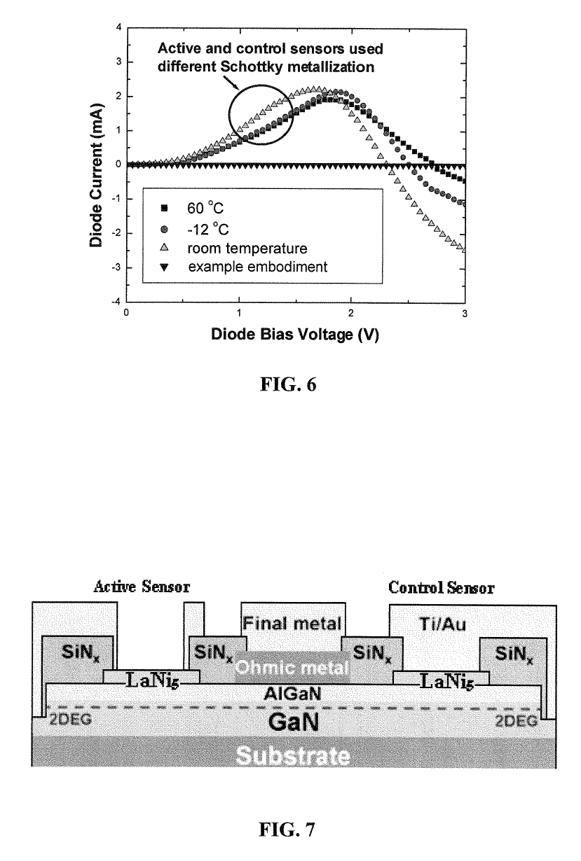

According to embodiments of the present invention, a normalized diode configuration is provided where the control device includes the same structure as the gate functionalization of the active device, but further includes the metal of a final metal layer, dielectrics, or polymers. For example, the active member of the normalized pair can be a Pt-based gate contact device and the control member of the normalized pair can be a Pt/Ti/Au, Pt/dielectric, or Pt/polymer based gate contact device. Similarly, if the active member is a Pd-based gate contact device, then the control member can be a Pd/Ti/Au, Pd/dielectric, or Pd/polymer based gate contact device. Advantageously, by providing both the control device and the active device with a same or similar gate functionalization (gate metal), Schottky characteristics exist for both devices. In addition, the inclusion of the Pt or Pd gate metal that is used for the active device on the control device brings the work function of the control device in line with the active device, thereby making the response to different temperature ambients in line with each other and reducing the effects of having different responses to the different temperature ambients. This effect can be seen in FIG. 6. FIG. 6 shows a plot of the difference of diode current vs. bias voltage between the reference (control) and the active sensor where Ti/Au is used as the gate contact instead of Pt/Ti/Au as used in accordance with an embodiment of the present invention.

Specifically, as shown in FIG. 6, by not providing both the control device and the active device with a same or similar gate functionalization, the device where Ti/Au is used as the gate contact shows sensitivity to temperature changes and applied bias. In contrast, an embodiment of the present invention using Pt/Ti/Au as the gate contact is capable of maintaining a constant current over change in bias and temperature. As shown in the plot, even where a control diode is used (but does not have the same gate metal as the active diode), the difference of the diode current from the control and active diode is not zero (normalized). This is due to the gate metal/semiconductor interface (semiconductor/Ti/Au) of the control sensor being different from the active diode (semiconductor/Pt). The work function of the Ti and Pt are different and the diode current of the Ti/Au based sensor and Pt based sensor would be different at different bias as well as different temperature. The Pt/Ti/Au based control sensor and the Pt based active sensor have the same metal contact/semiconductor interface, resulting in no difference of the diode current being observed.

It should be noted that these plots represent the current in an ambient environment having no hydrogen. Therefore, for the sensing device of embodiments of the present invention, the temperature and bias dependence of the response signal can be minimized and the signal be normalized to indicate only the presence of hydrogen.

For the active diode, the current increases upon introduction of the H₂ through a lowering of the effective barrier height. The H₂ catalytically decomposes on the Pt metallization and diffuses rapidly to the interface where it forms a dipole layer.

The normalized change in forward current or reverse current upon introduction of the hydrogen into the ambient environment is measurable.

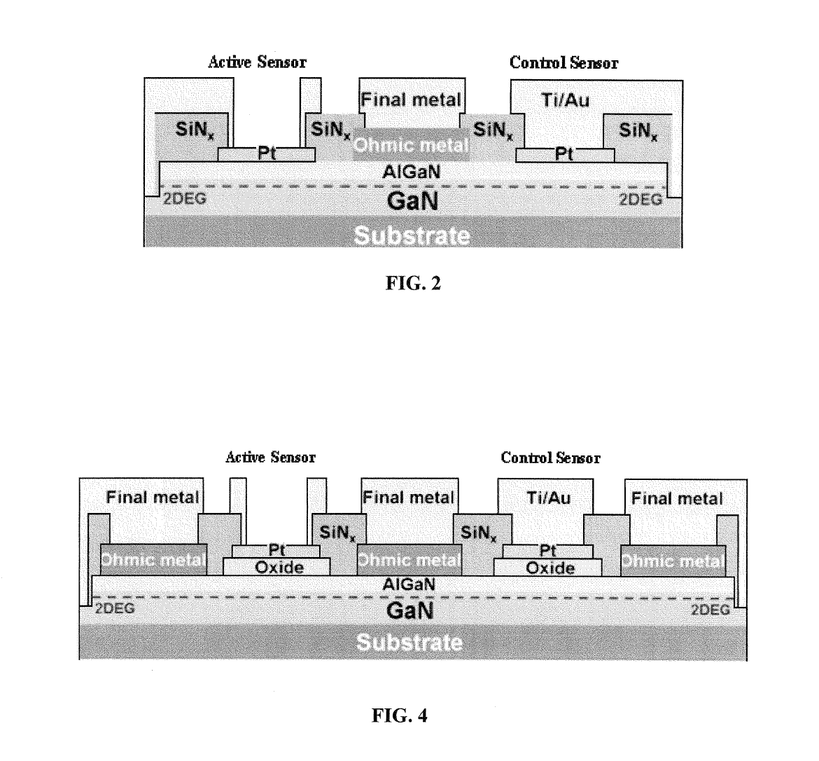

FIG. 2 shows a cross-sectional view of a hydrogen sensor according to an embodiment of the present invention. As shown in FIG. 2, the gate metal (in this case Pt) is formed directly on the gate region to provide diode-based characteristics. For normalization, the same gate metal functionalization is formed for both the control sensor device and active sensor device.

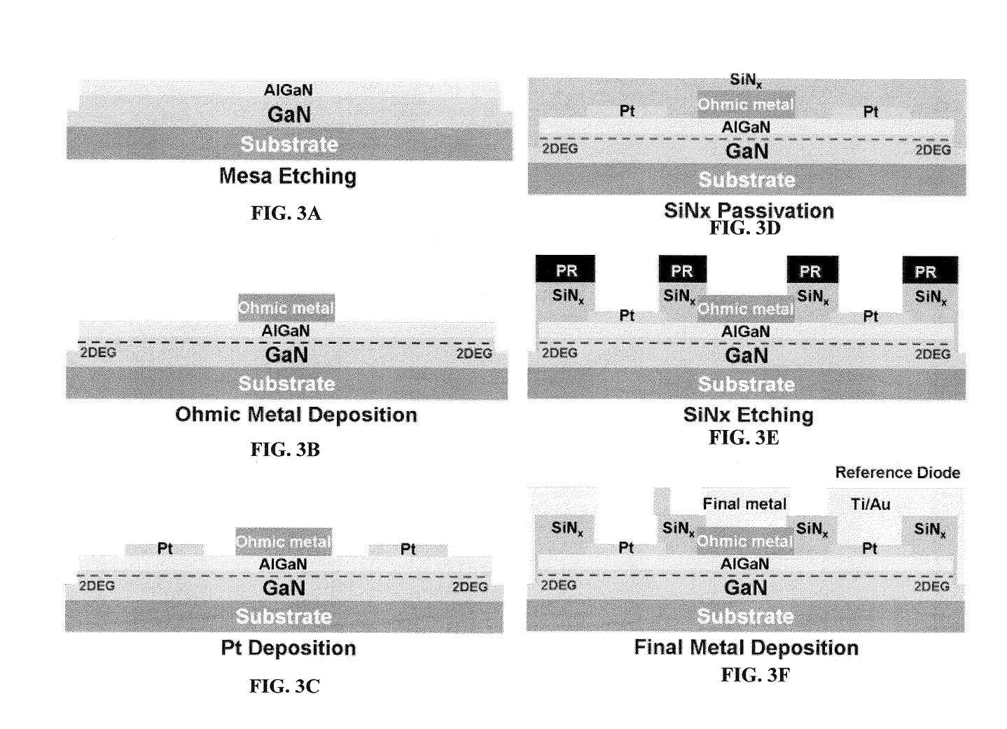

FIGS. 3A-3F show a fabrication process flow for a normalized hydrogen sensor pair according to an embodiment of the present invention. Referring to FIG. 3A, sensor regions can be defined through mesa etching a prepared substrate. The substrate can be any suitable substrate such as sapphire (Al₂O₃), silicon carbide (SiC), or silicon (Si). The substrate can be prepared with epitaxially grown layers of group III-IV elements. Deposition methods, such as Metal Organic Chemical Deposition (MOCVD) or molecular beam exitaxy (MBE), can be used to form the layers of the HEMT on the substrate. In one embodiment, the substrate can be prepared with AlGaN/GaN layers.

For the normalized arrangement, an active HEMT and a control HEMT are formed sharing a common contact. The common contact for the device can be formed through, for example, Ohmic metal deposition as shown in FIG. 3B. The common contact can be formed at a common source region between the two HEMTs (active and control). In one embodiment, the Ohmic metal deposition process can include titanium (Ti), aluminum (Al), platinum (Pt) and gold (Au) deposition. For example, the ohmic contacts can be formed by lift-off of e-beam deposited Ti/Al/Pt/Au and annealing at 700-900° C. for 30 seconds to 2 minutes under a flowing N₂ ambient environment. In another embodiment, the ohmic metal deposition process can use nickel (Ni), molybdenum (Mo), or iridium (Ir) in place of the Pt. Although not shown, the ohmic metal deposition process can also be used to form ohmic metal contacts for the drain regions of the HEMT devices.

Palladium can then be deposited and patterned to remain on the gate regions of the control and active sensor portions of the two HEMTs. In another embodiment as shown in FIG. 3C, platinum can be formed as the gate metal instead of Pd. A passivation layer can be formed on the substrate including on the gate metal, such as shown in FIG. 3D. The passivation layer can then be etched to open windows for final metal deposition on the gate regions and the common contact region (and the drain regions) of the device. In one embodiment, the passivation layer can be SiNx. Referring to FIG. 3E, an etch mask using a lithography process can be used to protect regions of the passivation layer while exposing the regions for final metal deposition. Once the gate metals and common electrode are exposed, a final metal deposition process can be performed. In one embodiment, the final metal can be Ti/Au. Then, the final metal layer can be etched to expose the gate metal at the active sensor portion while covering the control sensor portion as shown in FIG. 3F. The final metal layer is used as a final metal contact for the common contact. The final metal layer can also be used as the layer covering the gate metal on the control sensor region. For embodiments where a dielectric or polymer is used to cover the gate metal at the control sensor portion, the passivation layer formed as shown in FIG. 3D can remain on the gate metal at the control sensor portion when etching the windows for the depositing the final metal layer.

FIG. 4 shows a cross-sectional view of a hydrogen sensor according to an embodiment of the present invention. As shown in FIG. 4, the gate metal (in this case Pt/oxide) is formed on both the control and active sensor device to provide FET-based characteristics. As shown in FIG. 4, an ohmic contact metal can be formed at the drain region, and the functionalized gate region of the sensor can include a gate dielectric formed of an oxide, such as Sc₂O₃, through a contact window of a SiNx layer. For a normalized sensor arrangement, the ohmic contact metal can be formed for a common contact of the active and control sensors. In one embodiment, the oxide can be formed both in the contact window and on the SiNx layer. In other embodiments, the gate dielectric oxide can be HfO₂, Al₂O₃, Gd₂O₃, GGO, GGG, TiO₂, SiO₂, SiO, or SiNx. In an embodiment, before oxide deposition, the wafer can be exposed to ozone and heated in-situ at 300° C. cleaning for 10 min. A Schottky metal contact can be deposited on the top of the Sc₂O₃ in the gate region. As one example, the Schottky metal contact can be formed of Pt. Then, final metal of, for example, e-beam deposited Ti/Au interconnection contacts can be employed on the MOS-HEMTs.

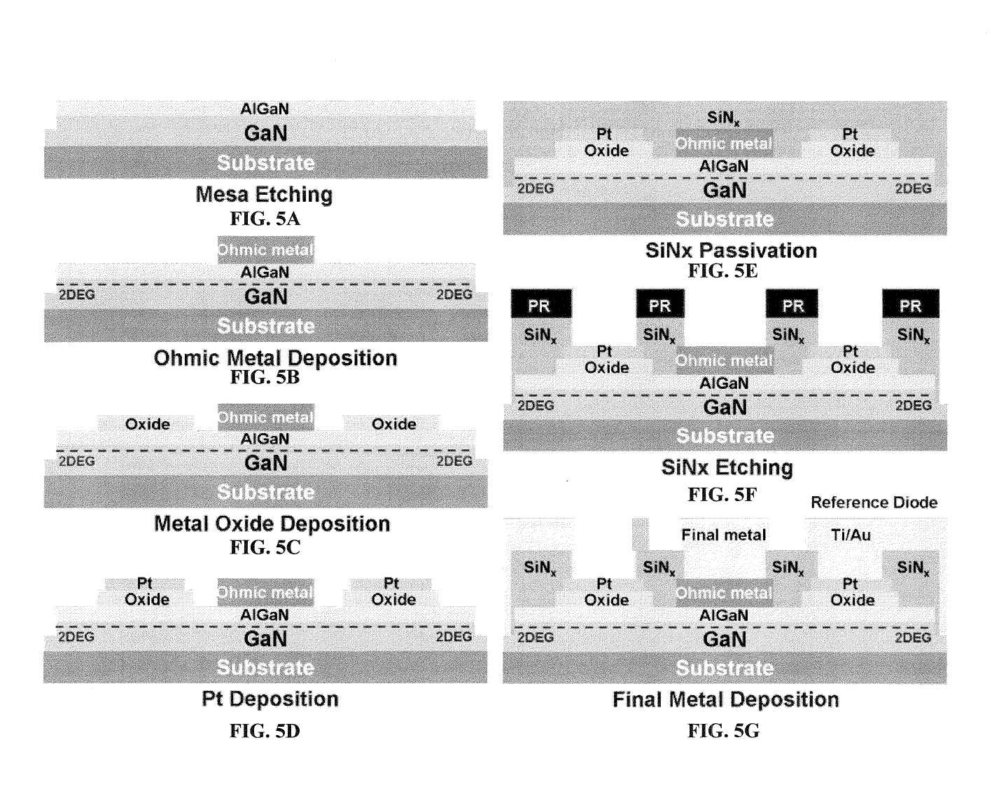

FIGS. 5A-5G show a fabrication process flow for a normalized hydrogen sensor pair according to another embodiment of the present invention. HEMT layer structures can be grown on a substrate. The substrate can be any suitable substrate such as Al₂O₃, SiC, or Si. Mesa etching can be conducted to define device regions as shown in FIG. 5A. For the normalized arrangement, an active HEMT and a control HEMT are formed sharing a common contact. The common contact can be formed at a common source region between the two HEMTs (active and control) by Ohmic metal deposition, such as shown in FIG. 5B. Although not shown in the drawings, this step can also form Ohmic contacts at the drain regions of the HEMTs. In one embodiment, the Ohmic metal deposition process can include Ti, Al, Pt, and Au deposition. For example, the ohmic contacts can be formed by lift-off of e-beam deposited Ti/Al/Pt/Au and annealing at 700-900° C. for 30 seconds to 2 minutes under a flowing N₂ ambient environment.

Differently from the embodiment shown in FIG. 3C, an oxide can be deposited on the gate regions of the control and active sensors to provide a MOS-HEMT configuration (see FIG. 5C). In one embodiment, the oxide can be a metal oxide. In a specific embodiment, the oxide can be Sc₂O₃. In one embodiment as shown in FIG. 5D, platinum can be deposited and patterned to remain on the oxide on the gate regions of the control and active sensor portions of the two HEMTs. In another embodiment, palladium can be deposited and patterned for the gate metal. A passivation layer can be formed on the substrate including on the platinum/oxide on the gate regions as shown in FIG. 5E. The passivation layer can then be etched to open windows for final metal deposition on the gate regions and the common contact region (and drain regions) of the device. In one embodiment, the passivation layer can be SiNx. As shown in FIG. 5F, an etch mask formed using a lithography process can be used to protect certain regions of the passivation layer while exposing other regions for final metal deposition. Once the gate metals and common electrode are exposed, a final metal deposition process can be performed. In one embodiment, the final metal can be Ti/Au. Then, the final metal layer can be etched to expose the gate metal region at the active sensor portion while covering the control sensor portion as shown in FIG. 5G. The final metal layer is used as a final metal contact for the common contact. Of course, embodiments are not limited to using the final metal for covering the gate metal of the control sensor portion. For example, a dielectric and/or a polymer can be used to cover the gate metal of the control sensor portion.

The oxide/gate metal normalized HEMT sensor can provide shortened recovery time as compared to the gate metal differential HEMT sensor.

Another embodiment of a hydrogen sensor is shown in FIG. 7. Referring to FIG. 7 LaNi₅ can be used on the gate region of a HEMT for hydrogen detection. A normalized design can also be applied to the LaNi₅-gated nitride HEMT for hydrogen sensing applications. In a further embodiment, a gate oxide can be included to provide FET-based characteristics.

In accordance with embodiments of the present invention, wide bandgap semiconductor sensors, such as nitride or silicon carbide based sensors, are amenable to low current applications because of their low intrinsic carrier concentrations and offer a wide range of temperature functionality. The ability of electronic devices fabricated in these materials to function in high temperature, high power and high flux/energy radiation conditions enables performance enhancements in a wide variety of spacecraft, satellite, homeland defense, mining, automobile, nuclear power and radar applications. Furthermore, with certain alterations in accordance with embodiments of the present invention, low temperature sensing can be accomplished.

AlGaN/GaN high electron mobility transistors (HEMTs) show promising performance for use in broad-band power amplifiers in base station applications due to the high sheet carrier concentration, electron mobility in the two dimensional electron gas (2DEG) channel and high saturation velocity. The high electron sheet carrier concentration of nitride HEMTs is induced by piezoelectric polarization of the strained AlGaN layer, and spontaneous polarization is very large in wurtzite III-nitrides. This provides an increased sensitivity relative to simple Schottky diodes fabricated on GaN layers. An additional attractive attribute of AlGaN/GaN diodes is the fact that gas sensors based on this material can be integrated with high-temperature electronic devices on the same chip. The advantages of GaN over SiC for sensing include the presence of the polarization-induced charge, the availability of a heterostructure and the more rapid pace of device technology development for GaN which is a popular material for commercialized light-emitting diode and laser diode businesses.

Although embodiments have been described with respect to AlGaN/GaN HEMTs, the invention is not limited thereto. Other nitride based or non-nitride based HEMTs can be utilized in accordance with embodiments of the present invention and within the scope of the subject invention.

According to an embodiment of the present invention, a normalized hydrogen sensor is provided having a control HEMT based hydrogen sensor and an active HEMT based hydrogen sensor connected with a common source. The control HEMT based sensor and the active HEMT based sensor are formed with a same gate metal to semiconductor interface. The control HEMT based sensor includes a final metal, dielectric, or polymer coating on the gate metal so as to inhibit exposure of the control HEMT based sensor to the ambient environment.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A hydrogen sensor comprising:
a first HEMT with functionalization comprising a material layer that selectively absorbs hydrogen gas; and
a second HEMT sharing a common source contact with the first HEMT, wherein the second HEMT has the functionalization comprising the material layer that selectively absorbs the hydrogen gas.

2. The hydrogen sensor according to claim 1, wherein the material layer that selectively absorbs hydrogen gas comprises palladium.

3. The hydrogen sensor according to claim 1, wherein the material layer that selectively absorbs hydrogen gas comprises platinum.

4. The hydrogen sensor according to claim 1, wherein the material layer that selectively absorbs hydrogen gas comprises LaNi₅.

5. The hydrogen sensor according to claim 1, further comprising an oxide layer below the material layer that selectively absorbs the hydrogen gas on the first HEMT and the second HEMT.

6. The hydrogen sensor according to claim 5, wherein the oxide layer comprises Sc₂O₃, HfO₂, Al₂O₃, Gd₂O₃, GGO (gallium gadolinium oxide), GGG (gallium gadolinium garnet), TiO₂, SiO₂, SiO, or SiNx.

7. The hydrogen sensor according to claim 1, further comprising a protective layer on the material layer that selectively absorbs the hydrogen gas of the second HEMT, wherein the protective layer inhibits exposure of the second HEMT to the hydrogen gas.

8. The hydrogen sensor according to claim 7, wherein the protective layer is a metal layer.

9. The hydrogen sensor according to claim 7, wherein the protective layer is a dielectric layer.

10. The hydrogen sensor according to claim 7, wherein the protective layer is a polymer layer.

11. A hydrogen sensor comprising:
a HEMT with gate functionalization comprising LaNi₅.

12. The hydrogen sensor according to claim 11, further comprising an oxide below the LaNi₅.

13. A method of hydrogen detection, comprising:
exposing to an environment, a hydrogen sensor comprising a first HEMT with functionalization comprising a material layer that selectively absorbs hydrogen gas; and a second HEMT sharing a common source contact with the first HEMT, wherein the second HEMT has the functionalization comprising the material layer that selectively absorbs the hydrogen gas.

14. The method of hydrogen detection according to claim 13, wherein the material layer that selectively absorbs hydrogen gas comprises palladium or platinum.

15. The method of hydrogen detection according to claim 13, wherein the material layer that selectively absorbs hydrogen gas comprises LaNi₅.

16. The method of hydrogen detection according to claim 13, wherein the hydrogen sensor further comprises an oxide layer below the material layer that selectively absorbs the hydrogen gas on the first HEMT and the second HEMT.

17. The method of hydrogen detection according to claim 16, wherein the oxide layer comprises Sc₂O₃, HfO₂, Al₂O₃, Gd₂O₃, GGO, GGG, TiO₂, SiO₂, SiO, or SiNx.

18. The method of hydrogen detection according to claim 13, wherein the hydrogen sensor further comprises a protective layer on the material layer that selectively absorbs the hydrogen gas of the second HEMT, wherein the protective layer inhibits exposure of the second HEMT to the hydrogen gas in the environment.

19. The method of hydrogen detection according to claim 18, wherein the protective layer is a metal layer.

20. The method of hydrogen detection according to claim 18, wherein the protective layer is a dielectric layer or a polymer layer.
